(19) United States
(12) Patent Application Publication
(10) Pub. No.: US 2021/0353563 A1
(43) Pub. Date: Nov. 18, 2021

(54) COMPOSITION AND METHOD FOR TREATMENT OF RESPIRATORY DISORDERS

(71) Applicant: Charles McDaniel, Pacific Pallsades, CA (US)

(72) Inventor: Charles McDaniel, Pacific Pallsades, CA (US)

(21) Appl. No.: 17/214,360

(22) Filed: Mar. 26, 2021

Related U.S. Application Data
(60) Provisional application No. 63/000,305, filed on Mar. 26, 2020.

(51) Int. Cl. A61K 31/138; A61K 45/06; A61K 9/00

(52) U.S. Cl. CPC A61K 31/138; A61M 11/00; A61K 9/007; A61K 45/06

(57) ABSTRACT

The present invention relates generally to the treatment of respiratory disorders. More particularly the invention is a composition and method for the treatment of respiratory disorders using inhaled norepinephrine reuptake inhibitor such as Atomoxetine.

### BACKGROUND OF THE INVENTION

The present invention relates generally to the treatment of respiratory disorders. More particularly the invention is a composition and method for the treatment of respiratory disorders using an inhaled norepinephrine reuptake inhibitor, for example Atomoxetine.

Atomoxetine is a norepinephrine reuptake inhibitor sold by Eli Lilly under the trade name Straterra®. Atomoxetine has the IUPAC chemical name 3R)—N-methyl-3-(2-methylphenoxy)-3-phenylpropan-1-amine; (R)—N-methyl-3-phenyl-3-(o-tolyloxy)-propan-1-amine, and was at one time also referred to as tomoxetine. Atomoxetine was originally developed as a proposed treatment for depression, however, that development was halted and it was subsequently developed for and is now sold as a treatment for ADHD (Attention Deficit Hyperactivity Disorder).

US Patent Application Publication No. 2006/0100290 proposed the use of orally administered Atomoxetine as a treatment of rhinitis and asthma. The publication proposed that Atomoxetine's inhibition of norepinephrine would function in an area of the brain responsible for regulation of the airways of the upper respiratory and lower respiratory tracts. It was thought that this would stabilize the epithelial membrane which lines these tracts to curb the transport of allergens across this membrane.

US Patent Application Publication No. 2004/0235925 proposed the use of a cyclooxygenase-2 selective inhibitor (COX-2 inhibitor) in combination with a norepinephrine reuptake inhibitor such as duloxetine, venlafaxine, Atomoxetine or combinations for the treatment of a CNS disorder and/or pain and inflammation or an inflammation-associated disorder.

US Patent Application Publication No. 2007/0219201 proposed the use of Atomoxetine in combination with a 5HT1_{A} receptor agonist for treatment of ADHD and related disorders, and for treatment of other central nervous system diseases or disorders.

### SUMMARY OF THE INVENTION

Atomoxetine has been previously proposed for use in the treatment of asthma and rhinitis by systemic administration. Systemic administration can lead to a number of drawbacks, which may be associated with the need to administer higher dosage, leading to increased total exposure that can cause enhanced or increased incidence of side effects. The function of Atomoxetine as a norepinephrine reuptake inhibitor does not discriminate as to the site of activity, so systemic administration can result in effects throughout the body, including throughout the Central Nervous System. It would be better to provide administration more directly to those areas of the body where treatment is required. For example, in respiratory conditions, treatment is preferentially focused locally in the smooth muscle of the respiratory tract, or respiratory tissue.

The prior art suggesting the possible use of Atomoxetine for respiratory and pain disorders has also focused on norepinephrine reuptake inhibitors for management of inflammation and inflammatory disorders. While inflammation may be associated with respiratory dysfunction, inflammation is not the only process that leads to respiratory dysfunction. Decreased respiratory function is also mediated by decreases in air passage diameter or cross-sectional volume. In some cases, decreased respiratory function can be mediated by smooth muscle or other contractile tissues via bronchoconstriction.

There are a number of prior art approaches to the treatment of respiratory disorders and several medications have been approved to treat bronchoconstriction. However, these medications likely operate through different mechanisms of action than Atomoxetine and can have deleterious side effects. Several β2 adrenergic receptor agonists have been used in the treatment of respiratory disorders. For example, Albuterol is a β2 adrenergic receptor agonist that has been used to treat bronchoconstriction, but is not effective for all patients. Further, cases of partial efficacy require adjunctive treatment, usually by supplementing treatment with inhaled or oral steroids. Albuterol, like other β2 agonists, is also not without side effects, including fine tremor, anxiety, headache, muscle cramps, dry mouth, palpitation, tachycardia, arrhythmia, flushing, and disturbances of sleep and behavior, which some patients find intolerable. It has relative contraindications for use in patients with seizure disorder, diabetes mellitus, hyperthyroidism, concurrent use of monoamine oxidase inhibitors or tricyclic antidepressants. Further, high doses or prolonged use may cause hypokalaemia, which is of concern especially in patients on diuretics. Steroids have also been used to treat respiratory disorders, likely through anti-inflammatory effects. However, the local and systemic side effects of steroid use are well known and may not be well tolerated in all patients. Inhaled use of norepinephrine reuptake inhibitors such as Atomoxetine according to the present invention would permit patients to minimize or eliminate β2 agonist exposure and minimize or eliminate steroid exposure. This would allow more optimal management of bronchoconstriction or other respiratory disorders.

In summary, the present invention is a method of treating a respiratory disorder by administering by inhalation a pharmaceutical composition having an effective amount of an active pharmaceutical ingredient, wherein the active pharmaceutical ingredient includes a norepinephrine reuptake inhibitor. An exemplary norepinephrine reuptake inhibitor for use with the invention is Atomoxetine. The respiratory disorder can be, for example, bronchoconstriction, exercise-induced bronchoconstriction (EIB), bronchospasm, EIB with asthma (EIA), asthma, Chronic Obstructive Pulmonary Disease (COPD), Chronic Obstructive Lung Disease, bronchitis, rhinitis and allergic rhinitis. According to the invention Atomoxetine or another norepinephrine reuptake inhibitor can be inhaled through the mouth or nose using, for example, an oral Metered Dose Inhaler (MDI), multi-channel inhaler Nasal spray, Nasal MDI, Nebulizer, Mist, or Mask.

Respiratory disorders treatable according to the invention can include inflammatory and non-inflammatory symptoms. The norepinephrine reuptake inhibitor such as Atomoxetine is present in an amount effective to treat the non-inflammatory symptoms, although inflammatory aspects may also be reduced with treatment. In some embodiments, the respiratory disorder is a non-inflammatory respiratory disorder, for example, a bronchoconstriction, such as exercise-induced bronchoconstriction.

In embodiments, the active pharmaceutical ingredient of the pharmaceutical composition consists essentially of a norepinephrine reuptake inhibitor such as Atomoxetine. In embodiments, the pharmaceutical composition also includes at least one pharmaceutically acceptable excipient.

Embodiments of the invention include an inhalable pharmaceutical composition having an active pharmaceutical ingredient and at least one other pharmaceutically acceptable excipient, in which the active pharmaceutical ingredient includes a norepinephrine reuptake inhibitor such as Atomoxetine. In some embodiments, the active pharmaceutical ingredient consists essentially of the norepinephrine reuptake inhibitor such as Atomoxetine. In other embodiments, the active pharmaceutical ingredient also includes a second active pharmaceutically agent. Examples of the second active pharmaceutically agent include, for example, mediators of inflammation, such as a steroidal and non-steroidal anti-inflammatory (NSAID), leukotriene receptor agonists, theophylline or other xanthines, cromolyn or other mast cell stabilizers, or monoclonal antibodies The pharmaceutical composition is provided in an inhalable form suitable for nasal or oral administration.

In other embodiments, the invention is an inhalation device containing an active pharmaceutical ingredient and at least one other pharmaceutically acceptable excipient, in which the active pharmaceutical ingredient comprises a norepinephrine reuptake inhibitor such as Atomoxetine. The device can be, for example, an oral metered dose inhaler, a nasal metered dose inhaler, nebulizer, mask, or a nasal spray bottle. The active pharmaceutical ingredient contained in the device is in a form suitable for administering as a spray or mist.

Further objectives and advantages, as well as the structure and function of preferred embodiments will become apparent from a consideration of the description herein.

### DETAILED DESCRIPTION OF THE INVENTION

Embodiments of the invention are discussed in detail below. In describing embodiments, specific terminology is employed for the sake of clarity. However, the invention is not intended to be limited to the specific terminology so selected. While specific exemplary embodiments are discussed, it should be understood that this is done for illustration purposes only. A person skilled in the relevant art will recognize that other components and configurations can be used without parting from the spirit and scope of the invention. All references cited herein are incorporated by reference as if each had been individually incorporated.

Atomoxetine is a norepinephrine reuptake inhibitor and generally acts to increase the effect and efficacy of norepinephrine by preventing reabsorption. When administered systemically, Atomoxetine acts generally on the CNS. Atomoxetine can also act directly on nerves and muscles to increase local availability of norepinephrine. By preventing norepinephrine uptake in a specific location proximal to where norepinephrine would be beneficial, local delivery of Atomoxetine effectively allows the norepinephrine to be present at the locus where it is needed. Norepinephrine can also be transported to nearby locations where its effect can be beneficial. However, systemic administration effectively dilutes local activity because it does not provide targeted delivery to the tissues and organs where it is needed.

While specific reference is made herein to Atomoxetine, it may be possible to utilize other norepinephrine reuptake inhibitors with similar effects and advantages. Other norepinephrine reuptake inhibitors include, for example, Amedalin, CP-39,332, Daledalin, Duloxetine, Edivoxetine, Esreboxetine, Lortalamine, Nisoxetine, Reboxetine, Talopram, Talsupram, Tandamine, Venlafaxine, and Viloxazine. Any of these can be substituted for Atomoxetine in compositions and methods of the present invention.

Many respiratory disorders involve bronchoconstriction either as a primary symptom, or often along with inflammation. There is some controversy in the art regarding whether inflammation plays a role in contributing to bronchoconstriction. Some literature suggests that, exercised-induced asthma (EIA) and exercise induced bronchoconstriction (EIB) are mediated by or associated with inflammation. (See Del Giacco, S. R., et al., Exercise and asthma: an overview. Eur Clin Respir J, 2015. 2: 27984 (Abstract) “Airways inflammation plays a fundamental role in EIA/EIB.”) However, as pointed out in the same article, EIA and EIB are terms that have inaccurately been used interchangeably, but are actually distinct respiratory illnesses, and although EIB can occur in patients with asthma, it also specifically states that EIB occurs in 10% of otherwise healthy individuals. Thus, the confusion in roles may be somewhat attributable to the inconsistent use of terminology.

At least one study concluded that “exercise-induced bronchoconstriction does not cause airway inflammation or airway hyperresponsiveness” after observing “that exercise-induced bronchoconstriction does not cause eosinophilic airway inflammation in subjects with asthma who develop airway inflammation with the same degree of allergen-induced bronchoconstriction.” (Gauvreau, G. M., et al., Exercise-induced bronchoconstriction does not cause eosinophilic airway inflammation or airway hyperresponsiveness in subjects with asthma. Am J Respir Crit Care Med, 2000. 162(4 Pt 1): p. 1302-7.) In evaluating prior studies, O'Byrne pointed out that “the fraction of exhaled nitric oxide (FEno) levels, which generally reflect airway inflammation, do not appear to correlate well with the development or severity of EIB”. (O'Byrne, P. M., Exercise-induced bronchoconstriction, in UpToDate, H. Hollingsworth, Editor. 2016, T. W. Post, Peter J. Barnes, Robert A. Wood, Helen Hollingsworth: Waltham, Mass.; www.UpToDate.com (accessed Feb. 23, 2016) It was also observed that “High-intensity exercise by patients with asthma can result in exercise-induced bronchoconstriction, partly due to leukotriene production, but it is still debated whether this causes airway inflammation.” (Gauvreau)

O'Byrne notes differences between EIB and EIA, that while the general population has 5-20% incidence of EIB, people with Asthma have EIB incidence of 90% stating that “the magnitude of EIB is most strongly correlated with the underlying degree of airway hyper responsiveness and the presence of airway inflammation, as measured by the number of airway eosinophils” (O'Byrne) pointing out that the magnitude of the bronchoconstriction is correlated with inflammation. But the fact that the two are correlated does not mean that they are the same thing. Gauvreau criticizes this study by casting doubt on the relationship between EIB and an increase in eosinophils, which were used as an indicator of inflammation, thus casting doubt on the relationship to the presence of eosinophils correlates with inflammation in EIB. (“This study has demonstrated that exercise-induced bronchoconstriction does not worsen eosinophilic airway inflammation or airway hyperresponsiveness in individuals with asthma who develop airway eosinophilic inflammation after allergen-induced bronchoconstriction.”) O'Byrne also offers data that refutes the correlation between inflammation and EIB. (“In contrast, the fraction of exhaled nitric oxide levels, which generally reflect airway inflammation, do not appear to correlate well with the development or severity of EIB.”)

A number of articles also describe the neurological basis of EIB, generally discussing how inflammatory mediators may regulate the degree to which the nervous system leads to smooth muscle constriction. Thus, while there are two processes that often work together they are also distinct processes. Sensory neurons have a role in controlling smooth muscle constriction—bronchoconstriction—and without the sensory neurons, bronchial constriction does not occur. (Trankner, D., et al., Population of sensory neurons essential for asthmatic hyperreactivity of inflamed airways. Proc Natl Acad Sci USA, 2014. 111(31): p. 11515-20.), i.e., sensory neurons are essential for bronchoconstriction. As such, the sensory neurons are a necessary component in the process of asthmatic hyperreactivity. However, asthma and inflammation is not necessary for bronchoconstriction, as 10%-20% of the general population has EIB but is otherwise health and without inflammation. (See Del Giacco and O'Byrne). Administered locally, a norepinephrine reuptake inhibitor such as Atomoxetine can act via the local nerves in a manner similar to its activity in the nerves (neurons) of the brain to permit locally available norepinephrine to act on smooth muscle resulting in airway dilation. As noted, what is clear is that not all patients suffering from asthma (which is inflammatory), are affected by EIB, which would be expected if EIB were purely an inflammatory disorder.

What does appear clear is that in many, if not all, respiratory disorders that involve airway constriction, non-inflammatory bronchoconstriction and inflammation coexist as distinct processes that can be triggered by the same or similar initial stimulus or insult, and can interact, but are not interchangeable conditions or treatable with the same modality. In particular, airway constriction and bronchoconstriction can result from smooth muscle contraction or constriction within the airways in which the muscle remains contracted or constricted. Thus, while anti-inflammatories may treat the inflammatory aspects of the respiratory disorders or conditions, the underlying constriction may not be adequately addressed. By locally providing Atomoxetine to respiratory tissues, availability of endogenous norepinephrine increases, acting locally on muscle tissues, for example smooth muscle or other contractile tissues, so that the muscles remain dilated. As a result, decreased respiratory function is ameliorated by an increase in air passage diameter or cross-sectional volume. Accordingly, dilation of the airways is provided, thereby treating the respiratory condition or disorder. Alternatively, for patients prone to constrictions of the airways, local administration of Atomoxetine according to the invention prior to the onset of symptoms can act prophylactically to prevent airway constriction.

The method of the present invention is preferred over existing or potential treatments that involve administration of epinephrine or norepinephrine. These treatments can effect additional nerve and muscle receptors and may be deleterious to pulmonary tissues. Furthermore, use of these treatments adds exogenous epinephrine or norepinephrine, which can have additional side effects and upset the balance of these compounds in the body. In contrast, rather than adding to the biological system, the present invention utilizes epinephrine or norepinephrine that are already present in the body, allowing the body to resorb and reuse the biologically produced compounds.

The present invention provides compositions and methods for the treatment of respiratory disorders. As used herein, the terms “treat,” “treatment,” etc. refer to any detectable, clinically significant improvement, delay in the onset, prevention of the onset, or amelioration of the disorder or any symptoms of a disorder or condition. Treatment does not require or demand a cure.

The invention provides a treatment of respiratory disorders by administering a pharmaceutical composition that includes an effective amount of an active pharmaceutical ingredient, where the active pharmaceutical ingredient includes at least one active pharmaceutically agent. As used herein, an active pharmaceutically agent is a single material or compound exerting a pharmaceutical effect. The active pharmaceutically agent may be identified as a member of a class of compounds with a particular effect, e.g. a norepinephrine reuptake inhibitor, or as a specific agent, for example, Atomoxetine. As will be understood in the art, a single material can at times include a complex mixture, for example, as with a biologic drug.

As used and described herein, respiratory disorders treatable according to the invention include not only diseases or temporary or chronic conditions of the respiratory system, but also respiratory distresses, especially constriction of the airways, that can arise as a secondary effect from some other condition, disorder, or stimulus. According to the present invention, localized administration of Atomoxetine to affected airways, for example the lungs, bronchia, as nasal passages, is accomplished through oral or nasal inhalation.

More particularly, the invention provides a treatment of respiratory disorders by administering a pharmaceutical composition that includes an effective amount of an active pharmaceutical ingredient, where the active pharmaceutical ingredient includes a norepinephrine reuptake inhibitor. A particular exemplary norepinephrine reuptake inhibitor is Atomoxetine. In some embodiments of the invention, Atomoxetine is the only active pharmaceutically agent in the active pharmaceutical ingredient. In other embodiments, the active pharmaceutical ingredient can include other active pharmaceutically agents, for example, mediators of inflammation, such as a steroidal and non-steroidal anti-inflammatories (NSAID), leukotriene receptor agonists, theophylline or other xanthines, cromolyn or other mast cell stabilizers, or monoclonal antibodies. In addition to the active pharmaceutical ingredient, the pharmaceutical composition can include other excipients or additives in a formulation suitable for administration by inhalation. As used here “suitable for administration by inhalation” means that the composition is in a form that can be contained within a device designed to administer an inhalable composition. Forms suitable for administration by inhalation are known in the art and may be, for example, an aerosol designed to be dispensed from a pressurized container, or a liquid, for example to be dispensed as a saline solution or dispersion as in, for example, a nasal spray or nebulizer.

Atomoxetine used in pharmaceutical compositions according to the invention can be present in a free form or as a pharmaceutically acceptable salt together with one or more pharmaceutically acceptable excipients such as carriers or diluents. Any conventional technique may be used for the preparation of pharmaceutical compositions according to the invention. As used herein, “excipients” include, but are not limited to, additional ingredients such as one or more of the following: surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; pharmaceutically acceptable polymeric or hydrophobic materials as well as other components

Although the descriptions of pharmaceutical compositions provided herein are principally directed to pharmaceutical compositions which are suitable for administration to humans, it will be understood by the skilled artisan, based on this disclosure, that such compositions are generally suitable for administration to any mammal. Preparation of compositions suitable for administration to various animals is well understood, and the ordinarily skilled veterinary pharmacologist can design and perform such modifications with routine experimentation based on pharmaceutical compositions for administration to humans.

Liquid formulations of a pharmaceutical composition of the invention which are suitable for administration may be prepared, packaged, and sold either in liquid form or in the form of a dry product intended for reconstitution with water or another suitable vehicle prior to use.

Liquid suspensions, in which the active ingredient is dispersed in an aqueous or oily vehicle, and liquid solutions, in which the active ingredient is dissolved in an aqueous or oily vehicle, may be prepared using conventional methods or methods to be developed. Liquid suspension of the active ingredient may be in an aqueous or oily vehicle and may further include one or more additional components such as, for example, suspending agents, dispersing or wetting agents, emulsifying agents, demulcents, preservatives, buffers, salts, flavorings, coloring agents, and sweetening agents. Oily suspensions may further comprise a thickening agent. Liquid solutions of the active ingredient may be in an aqueous or oily vehicle and may further include one or more additional components such as, for example, preservatives, buffers, salts, flavorings, coloring agents, and sweetening agents.

Pharmaceutical compositions according to the present invention may contain, for example, an amount of Atomoxetine suitable to deliver from about 0.5 mg to 100 mg of Atomoxetine per dose or from about 1 mg to 25 mg Atomoxetine per dose, or from about 2 mg to about 10 mg per dose. In other embodiments, the pharmaceutical compositions may contain, for example, an amount of Atomoxetine suitable to deliver from about 0.5 mg to 100 mg of Atomoxetine per day or from about 1 mg to 25 mg Atomoxetine per day, or from about 2 mg to about 10 mg per day, where the daily dose is separated into 1 to 6 doses. The composition can be administered via an inhaled dose that can include 1-2 puffs per inhalations, and inhalations can occur every 4-24 hours (1-6 times per day) or as needed. Other norepinephrine reuptake inhibitors can be administered at appropriate dosages to achieve a similar effect. One skilled in the art can determine the appropriate dose and schedule in order to achieve the desired effective amount or effective concentration of the agent in the individual patient. The dose of a composition of the invention, administered to an animal, particularly a human, in the context of the present invention should be sufficient to effect at least a detectable amount of a diagnostic or therapeutic response in the individual over a reasonable time frame. The dose used to achieve a desired effect will be determined by a variety of factors, including the potency of the particular agent being administered, the pharmacodynamics associated with the agent in the host, the severity of the disease state of infected individuals, other medications being administered to the subject, etc. The size of the dose also will be determined by the existence of any adverse side effects that may accompany the particular agent, or composition thereof, employed. It is generally desirable, whenever possible, to keep adverse side effects to a minimum.

Devices in which the pharmaceutical composition may be contained include, for example, an oral metered dose inhaler (MDI), multi-channel inhaler, nasal spray, nasal mdi, nebulizer, mist, or mask. A multichannel inhaler can be, for example, a metered dose inhaler containing multiple compartments, each or which can contain a separate pharmaceutically active ingredient, allowing for administration of multiple pharmaceutically active ingredients without the need to premix the two components. In some embodiments, the device is designed to provide a reproducible standardized dose, as can be accomplished with, for example, a metered dose inhaler. In some embodiments, the invention is a filled device that includes an inhalation device and the pharmaceutical composition that includes an effective amount of an active pharmaceutical ingredient, where the active pharmaceutical ingredient includes Atomoxetine, according to any of the embodiments of the pharmaceutical composition as described above.

The compositions and methods described herein can be used to treat most respiratory disorders that involve constriction of airways. In some embodiments, any of the compositions and methods can be used to treat a non-inflammatory respiratory disorder. In other embodiments, compositions and methods can be used to treat a respiratory disorder having both inflammatory and non-inflammatory symptoms. In treating a disorder with inflammatory and non-inflammatory symptoms, the present invention preferentially addresses the non-inflammatory aspects or symptoms although, as will be appreciated by persons in the art, treating the non-inflammatory aspects may have beneficial effects in treating the inflammatory aspects as well. Examples of respiratory disorders treatable according to the invention include, for example, respiratory illnesses, acute or chronic conditions of the respiratory track, acute or chronic condition of the respiratory system, acute or chronic disorders of the respiratory track, and acute or chronic disorders of the respiratory system. Specific disorders can include exercise-induced bronchoconstriction (EIB), Bronchoconstriction associated with other conditions, Bronchospasm associated with other conditions, EIB with asthma (aka EIA), Asthma, Chronic Obstructive Pulmonary Disease, Chronic Obstructive Lung Disease, Bronchitis, Rhinitis and allergic rhinitis. EIB is a particular condition amenable to treatment according to the present invention.

As used herein, respiratory disorders includes not only specific disorders themselves, but other conditions which involve airway constriction. Many of these conditions involve both inflammation related symptoms and non-inflammation related symptoms. According to the present invention, inhaled Atomoxetine can help alleviate airway constriction, particularly airway constriction that is non-inflammatory in origin. Conditions which can cause airway constriction and can beneficially be treated with relief of airway constriction and bronchoconstriction can include, for example, neuromuscular disorders, thoracic restrictive disease, obesity-hypoventilation syndrome, obstructive lung disease, illnesses, or processes, restrictive lung disease, illnesses, or processes, vascular disease, illnesses, or processes, infection, neonatal respiratory disease, illnesses, or processes, tumor, pleural cavity diseases, Gastroesophageal Reflux Disease (GERD) and other respiratory processes occurring secondary to other disease or illness, infection, medication or drug, trauma, high altitude, toxin, or exposure.

The present compositions and methods can also be used to treat respiratory disorders where inflammation occurs but also have elements that are likely non-inflammatory. In some disorders, the disease is primarily inflammatory, but the inflammation associated with the disease is not the cause of the respiratory impairment, it is some other consequence of the disease that effects the respiratory system. For example, in Rheumatoid Arthritis and Scleroderma, it is the subsequent fibrosis that occurs along with the disease that results in respiratory impairment. Such conditions that may have non-inflammatory as well as inflammatory processes include, for example chronic obstructive pulmonary disease (COPD), Emphysema, Acute Bronchitis, Chronic Bronchitis, Bronchiolitis, bronchiolitis obliterans with organizing pneumonia, Bronchiectasis, Broncopulmonary dysplasia, Cystic Fibrosis, Pneumoconiosis (e.g. Asbestosis), Radiation fibrosis, pulmonary fibrosis, Rheumatoid arthritis, Sarcoid or Scleroderma, Hypersensitivity pneumonitis (due to allergy to inhaled particles), Acute Respiratory Distress Syndrome (ARDS), Infant respiratory distress syndrome, TB, Idiopathic pulmonary fibrosis, Interstitial lung disease, Idiopathic interstitial pneumonia, Sarcoidosis, Eosinophilia pneumonia, Lymphangioleimomyomatosis, Pulmonary Langerhans' cell histiocytosis, Pulmonary Alveolar Proteinosis, Nonmuscular diseases of upper thorax—kyphosis, pectus carinatum, pectus excavatum, Diseases of lower thoracic abdomen such as obesity, diaphragmatic hernia, ascites, and Pleural thickening.

The present invention is also useful for treating respiratory disorders triggered by other causes, for example non-inflammatory irritants or processes. As an example, exercise can trigger non-inflammatory bronchoconstriction. Inhalation of toxic fumes or irritants can trigger bronchoconstriction as well. Temperature change, cold air, warm air, stretching of the airway passages can all trigger bronchoconstriction. This can be followed by inflammation, although not necessarily or typically from the bronchoconstriction, but from trauma to the surrounding tissue caused by the toxin or irritant. Various kinds of trauma can also cause bronchoconstriction, with inflammation often occurring as well either along with or after bronchoconstriction. In chronic inflammatory processes, nerve receptors can become sensitized by inflammation and bronchoconstriction occurs at a lower threshold. Persons skilled in the art will recognize similar conditions that can result in a respiratory disorder.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

What is claimed is:

1. A method of treating a respiratory disorder comprising administering by inhalation a pharmaceutical composition to a person in need thereof, wherein the pharmaceutical composition comprises an effective amount of an active pharmaceutical ingredient, and wherein the active pharmaceutical ingredient comprises a norepinephrine reuptake inhibitor.

2. The method of claim 1, wherein the norepinephrine reuptake inhibitor is Atomoxetine.

3. The method of claim 1, wherein the respiratory disorder is selected from the group consisting of bronchoconstriction, exercise-induced bronchoconstriction (EIB), bronchospasm, EIB with asthma (EIA), asthma, Chronic Obstructive Pulmonary Disease (COPD), Chronic Obstructive Lung Disease, bronchitis, rhinitis and allergic rhinitis.

4. The method of claim 1, wherein inhalation is through the mouth or nose.

5. The method of claim 1, wherein the administration utilizes an oral Metered Dose Inhaler (MDI), multi-channel inhaler Nasal spray, Nasal MDI, Nebulizer, Mist, or Mask.

6. The method of claim 1, wherein the respiratory disorder comprises inflammatory and non-inflammatory symptoms and the norepinephrine reuptake inhibitor is present in an amount effective to treat the non-inflammatory symptoms.

7. The method of claim 1, wherein the respiratory disorder is a non-inflammatory respiratory disorder.

8. The method of claim 7, wherein the non-inflammatory respiratory disorder is bronchoconstriction.

9. The method of claim 7, wherein the non-inflammatory respiratory disorder is EIB.

10. The method of claim 2, wherein the active pharmaceutical ingredient consists essentially of Atomoxetine.

11. The method of claim 10, wherein the pharmaceutical composition further comprises at least one pharmaceutically acceptable excipient.

12. An inhalable pharmaceutical composition comprising an active pharmaceutical ingredient and at least one other pharmaceutically acceptable excipient, wherein the active pharmaceutical ingredient comprises a norepinephrine reuptake inhibitor.

13. The inhalable pharmaceutical composition of claim 12, wherein the active pharmaceutical ingredient consists essentially of the norepinephrine reuptake inhibitor.

14. The inhalable pharmaceutical composition of claim 12, wherein the norepinephrine reuptake inhibitor is Atomoxetine.

15. The inhalable pharmaceutical composition of claim 12, wherein the active pharmaceutical ingredient further comprises a second active pharmaceutical agent.

16. The inhalable pharmaceutical composition of claim 15, wherein the second active pharmaceutical agent is selected from mediators of inflammation, such as steroidal and non-steroidal anti-inflammatories (NSAID), leukotriene receptor agonists, theophylline or other xanthines, cromolyn or other mast cell stabilizers, and monoclonal antibodies.

17. The inhalable pharmaceutical composition of claim 12, wherein the inhalable pharmaceutical composition is in a form suitable for nasal or oral administration.

18. A device comprising an inhalation device containing an active pharmaceutical ingredient and at least one other pharmaceutically acceptable excipient, wherein the active pharmaceutical ingredient comprises Atomoxetine.

19. The device of claim 18, wherein the inhalation device is an oral metered dose inhaler, a nasal metered dose inhaler, nebulizer, mask, or a nasal spray bottle.

20. The method of claim 18, wherein the active pharmaceutical ingredient is in a composition suitable for administering as a spray or mist.
